(19) United States
(12) Patent Application Publication
(10) Pub. No.: US 2025/0152089 A1
(43) Pub. Date: May 15, 2025

(54) MEASUREMENT OF TISSUE VIABILITY

(71) Applicant: BBI MEDICAL INNOVATIONS, LLC, Los Angeles, CA (US)

(72) Inventors: Martin F. BURNS, Los Angeles, CA (US); Sara BARRINGTON, Thousand Oaks, CA (US); Graham O. ROSS, Oceanside, CA (US)

(73) Assignee: BBI MEDICAL INNOVATIONS, LLC, Los Angeles, CA (US)

(21) Appl. No.: 19/027,982

(22) Filed: Jan. 17, 2025

Related U.S. Application Data
(63) Continuation of application No. 17/692,926, filed on Mar. 11, 2022.
Application No. 17/692,926 is a division of application No. 15/887,837, filed on Feb. 2, 2018, now Pat. No. 11,304,652.
(60) Provisional application No. 62/521,926, filed on Jun. 19, 2017.
(60) Provisional application No. 62/454,487, filed on Feb. 3, 2017.

(51) Int. Cl. A61B 5/00; A61B 5/0531; A61B 5/0537; G01R 27/26

(52) U.S. Cl. CPC A61B 5/445; A61B 5/0531; A61B 5/0537; A61B 5/4878; G01R 27/2605

(57) ABSTRACT

The present disclosure provides apparatuses and methods for measuring sub-epidermal moisture as an indication of tissue viability and providing information regarding the location of a boundary of non-viable tissue.

### CROSS-REFERENCE TO RELATED APPLICATIONS

This application is a continuation of U.S. application Ser. No. 17/692,926 filed Mar. 11, 2022, which is a divisional of U.S. application Ser. No. 15/887,837 filed Feb. 2, 2018 (now U.S. Pat. No. 11,304,652), which claims the benefit of priority of U.S. Provisional Application 62/454,487 filed Feb. 3, 2017, and U.S. Provisional Application 62/521,926 filed Jun. 19, 2017, each of which is herein incorporated by reference in its entirety.

### FIELD

The present disclosure provides apparatus and methods for measuring type and degree of tissue damage around a burn or other type of wound.

### BACKGROUND

Serious wounds and burns may have regions of various degrees of damage surrounding the wound site. Effective treatment may require removal of non-viable tissue, yet it can be difficult to visually assess tissue viability. For an open wound such as a burn, there may be a region of non-viable tissue around the immediate wound while further away the tissue may be less damaged and characterized by swelling known as “edema” yet viable and likely to recover.

A common method of burn evaluation assesses the visual and tactile characteristics, namely wound appearance, capillary blanching and refill, capillary staining, and burn wound sensibility to light touch and pinprick. Estimation of the burn depth is difficult. In addition, burn wounds are dynamic and can progress over time and the changes do not immediately become visually apparent.

### SUMMARY

In an aspect, the present disclosure provides for, and includes, an apparatus for mapping areas of damage around a wound, the apparatus comprising: a plurality of electrodes embedded on a substrate configured to be placed over an area of tissue that includes the wound, where combinations of the electrodes are capable of forming a plurality of virtual capacitive sensors and each of the virtual capacitive sensors is configured to measure a capacitance of a region of tissue proximate to the respective virtual capacitive sensor, a plurality of visual indicators embedded on the substrate, a drive circuit electronically coupled to the electrodes and visual indicators, a processor electronically coupled to the drive circuit, and a non-transitory computer-readable medium electronically coupled to the processor and comprising instructions stored thereon that, when executed on the processor, perform the steps of: receiving information regarding the measured capacitance from a subset of the plurality of virtual capacitive sensors via the drive circuit, determining a boundary between viable and non-viable tissue, and activating via the drive circuit a portion of the plurality of visual indicators to indicate the boundary.

In an aspect, the present disclosure provides for, and includes, an apparatus for determining a depth of a burn wound, the apparatus comprising: a pair of electrodes capable of forming a capacitive sensor that is configured to measure a capacitance of a region of tissue proximate to the pair of electrodes, a drive circuit electronically coupled to the capacitive sensor, a processor electronically coupled to the drive circuit, and a non-transitory computer-readable medium electronically coupled to the processor and comprising instructions stored thereon that, when executed on the processor, perform the steps of: receiving information regarding the measured capacitance from the capacitive sensor via the drive circuit, comparing the information to a data array comprising pairs of capacitances and depths of burns, and determining the depth of the burn wound associated with the measured capacitance.

In an aspect, the present disclosure provides for, and includes, an apparatus for mapping areas of damage around a wound, the apparatus comprising: a plurality of electrodes embedded on a substrate configured to be placed over a portion of an area of tissue that includes the wound, where pairs of the electrodes are capable of forming a capacitive sensor that is configured to measure a capacitance of a region of tissue proximate to the capacitive sensor, a projector capable of projecting a visual indicator onto the area of tissue that includes the wound, a drive circuit electronically coupled to the plurality of electrodes and the projector, a processor electronically coupled to the drive circuit, and a non-transitory computer-readable medium electronically coupled to the processor and comprising instructions stored thereon that, when executed on the processor, perform the steps of: receiving information regarding the measured capacitance from one or more of the formed capacitive sensors, determining a first boundary between a first type of tissue and a second type of tissue, and causing the projector to project the visual indicator to indicate the boundary.

In one aspect, the present disclosure provides for, and includes a method for mapping areas of damage around a wound, the method comprising: obtaining capacitance measurements over an area of a tissue including the wound using a plurality of electrodes; converting each measured capacitance to an associated sub-epidermal moisture (SEM) value; and marking a first boundary encompassing regions of tissue associated with SEM values that are lesser than a first threshold.

### BRIEF DESCRIPTION OF THE DRAWINGS

Some aspects of the disclosure are herein described, by way of example only, with reference to the accompanying drawings. With specific reference now to the drawings in detail, it is stressed that the particulars shown are by way of example and are for purposes of illustrative discussion of aspects of the disclosure. In this regard, the description and the drawings, considered alone and together, make apparent to those skilled in the art how aspects of the disclosure may be practiced.

FIG. 1A discloses a toroidal bioimpedance sensor.

FIG. 1B discloses an idealized field map created by the toroidal sensor of FIG. 1A when activated.

FIG. 1C discloses a SEM scanner that comprises the sensor of FIG. 1A.

FIG. 2 is a first exemplary array of electrodes.

FIG. 3 is an exemplary array of electrodes according to the present disclosure.

FIG. 4A illustrates a first example of how the array of electrodes disclosed in FIG. 3 is configured to form a bioimpedance sensor according to the present disclosure.

FIG. 4B illustrates a second example of how the array of electrodes disclosed in FIG. 3 is configured to form a bioimpedance sensor according to the present disclosure.

FIG. 5A depicts an example 3^{rd}-degree burn with an open wound.

FIG. 5B depicts a cross-section of the wound of FIG. 5A.

FIG. 6 provides an example plot 600 of how SEM values may vary across the wound of FIG. 5A, according to the present disclosure.

FIG. 7 discloses a first exemplary aspect of an SEM sensing apparatus according to the present disclosure.

FIG. 8A discloses a second exemplary aspect of an SEM sensing apparatus according to the present disclosure.

FIG. 8B discloses a third exemplary aspect of an SEM sensing apparatus according to the present disclosure.

FIG. 9 discloses an aspect of an apparatus for mapping areas of damage according to the present disclosure.

### DETAILED DESCRIPTION

The present disclosure describes measurement of various electrical characteristics and derivation of SEM values indicative of the accumulation or depletion of extracellular fluid (ECF), also referred to as intercellular fluid, and the application of this information to the assessment of tissue viability. Examples are provided of application to thermal burns yet are applicable to other types of wounds. These examples are not limiting and the demonstrated principles may be applied to a larger scope of injuries and conditions than the specific example. For example, apparatus and methods disclosed in relation to a 3^{rd}-degree burn may be used with equal efficacy to an open cut, gangrene, an ulcer, or other similar injury.

Assessment of tissue viability around wounds and burns may be improved by determination of the amount of SEM in the tissue surrounding the actual damage. Typically, the tissue immediately around a wound will exhibit a reduced level of SEM, indicating a lower level of tissue viability. Further out from the wound, the tissue will exhibit an increased level of moisture, or edema. This value may be very high around the edge of the low-moisture tissue, indicating a high degree of damage with a high risk of eventual tissue death. The SEM value may taper off with increasing distance from the wound, where a moderately raised SEM level indicates damage with a higher chance of tissue viability. Mapping the areas of low-viability tissue, as indicated by reduced levels of tissue moisture, and the surrounding area of edema can provide important guidance to a clinician during the treatment of the wound.

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular embodiments of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments or aspects only and is not intended to be limiting of the disclosure.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

U.S. patent applications Ser. Nos. 14/827,375 and 15/134,110 are incorporated herein by reference in their entirety.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The methods disclosed herein comprise one or more steps or actions for achieving the described method. The method steps and/or actions may be interchanged with one another without departing from the scope of the present invention. In other words, unless a specific order of steps or actions is required for proper operation of the embodiment, the order and/or use of specific steps and/or actions may be modified without departing from the scope of the present invention.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a length, a frequency, or a SEM value and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The terms “comprise,” “comprises,” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

As used herein, the term “sub-epidermal moisture” or “SEM” refers to the increase in tissue fluid and local edema caused by vascular leakiness and other changes that modify the underlying structure of the damaged tissue in the presence of continued pressure on tissue, apoptosis, necrosis, and the inflammatory process.

As used herein, a “system” may be a collection of devices in wired or wireless communication with each other.

As used herein, “interrogate” refers to the use of radiofrequency energy to penetrate into a patient's skin.

As used herein, a “patient” may be a human or animal subject.

As used herein, a “3^{rd}-degree burn” refers to a full thickness burn that goes through the dermis and affect deeper tissues.

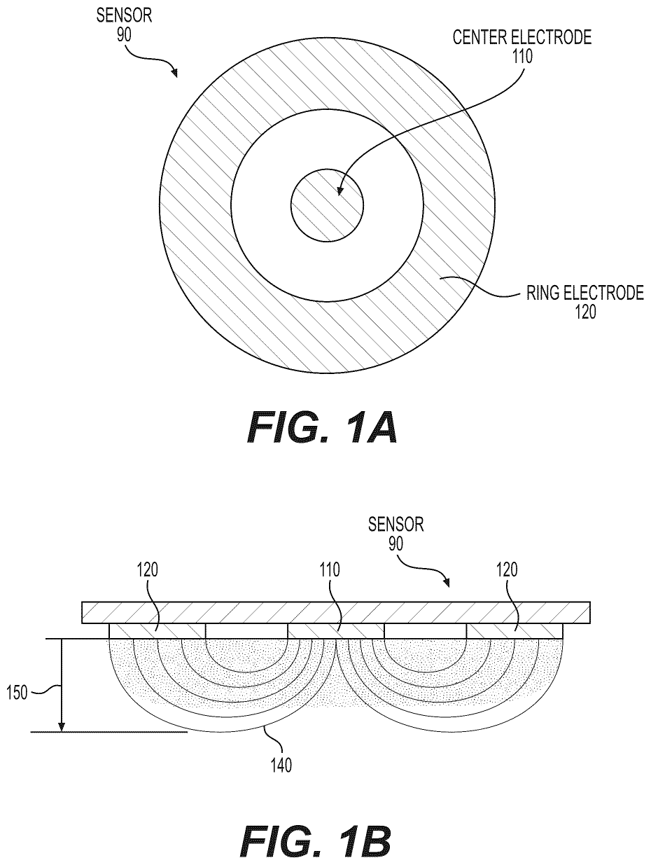

FIG. 1A discloses a toroidal bioimpedance sensor 90. In this exemplary configuration, a center electrode 110 is surrounded by a ring electrode 120. Without being limited to a particular theory, a gap between two electrodes of sensor 90 can affect the depth of field penetration into a substrate below sensor 90. In an aspect, a ground plane (not visible in FIG. 1A), is parallel to and separate from the plane of the electrodes. In one aspect, a ground plan extends beyond the outer diameter of ring electrode 120. Without being limited to a particular theory, a ground plane can limit the field between electrodes 110 and 120 to a single side of the plane of electrodes 110 and 120 that is on the opposite side of the plane of electrodes 110 and 120 from the ground plane.

FIG. 1B discloses an idealized field map created by a toroidal sensor of FIG. 1A when activated by a drive circuit (not shown in FIG. 1B). In one aspect, when an electric voltage is applied across two electrodes 110, 120, an electric field 140 is generated between electrodes 110 and 120 that extends outward from the plane of electrodes 110 and 120 to a depth of field 150. In an aspect, the diameter of a center electrode 110, the inner and outer diameters of a ring electrode 120, and the gap between two electrodes 110 and 120 may be varied to change characteristics of field 140, for example the depth of field 150.

In use, a drive circuit can measure an electrical property or parameter that comprises one or more of a resistance, a capacitance, an inductance, an impedance, a reluctance, or other electrical characteristic as sensed by electric field 140. Depending on the type of drive circuit being employed in an apparatus, a sensor of an apparatus may be a bipolar radiofrequency sensor, a bioimpedance sensor, a capacitive sensor, or an SEM sensor. In an aspect, the measured electrical parameter is related to the moisture content of the epidermis of a patient at a depth that is determined by the geometry of electrodes 110 and 120, the frequency and strength of electrical field 140, and other operating characteristics of an apparatus drive circuit. In one aspect, the measured moisture content is equivalent to the SEM content with a value on a predetermined scale. In an aspect, a predetermined scale may range from 0 to 20, such as from 0 to 1, from 0 to 2, from 0 to 3, from 0 to 4, from 0 to 5, from 0 to 6, from 0 to 7, from 0 to 8, from 0 to 9, from 0 to 10, from 0 to 11, from 0 to 12, from 0 to 13, from 0 to 14, from 0 to 15, from 0 to 16, from 0 to 17, from 0 to 18, from 0 to 19. In one aspect, a predetermined scaled can be scaled by a factor or a multiple based on the values provided herein.

FIG. 1C provides top and bottom views of a SEM scanner 170 that comprises electronics that drive sensor 174, which is similar to sensor 90 of FIG. 1A, and measure a capacitance between electrodes 110 and 120. This capacitance is converted to a SEM value that is displayed on display 176.

These aspects of sensor 90 and SEM scanner 170 are disclosed in WO 2016/172263, from which the U.S. patent application Ser. No. 15/134,110 was filed as a national phase entry.

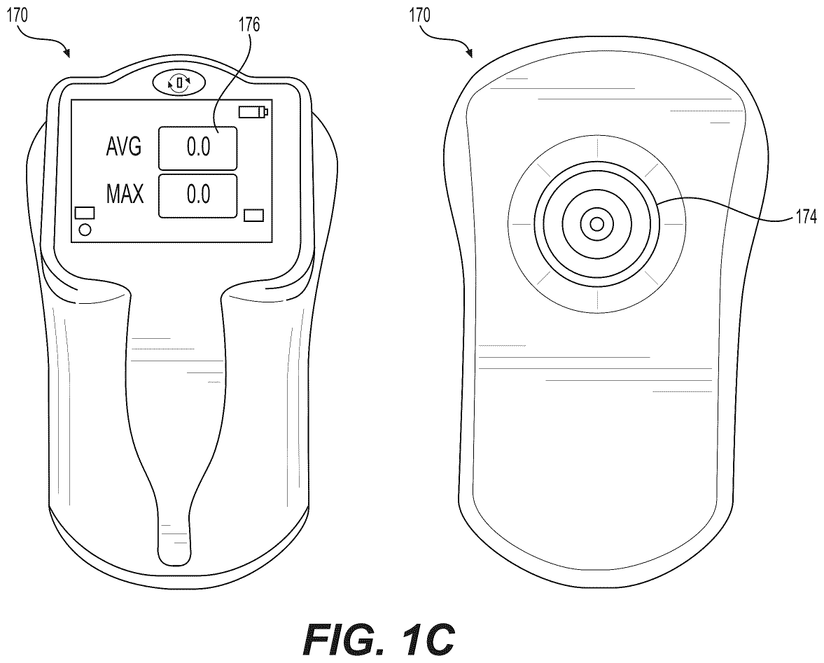

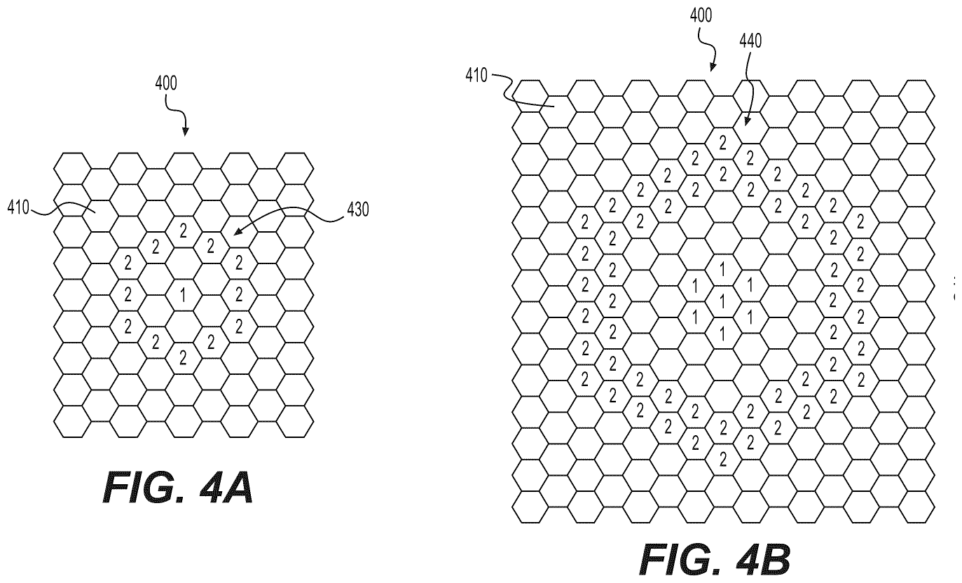

FIG. 2 depicts an exemplary electrode array 290, according to the present disclosure. In an aspect, an array 290 is composed of individual electrodes 300 disposed, in this example, in a regular pattern over a substrate 292. In an aspect, each electrode 300 is separately coupled (through conductive elements not shown in FIGS. 2 through 4B) to a circuit, such as described with respect to FIG. 4A, that is configured to measure an electrical parameter. In one aspect, a “virtual sensor” is created by selective connection of predetermined subsets of electrodes 300 to a common element of a circuit. In one aspect, a particular electrode 310 is connected as a center electrode, similar to electrode 110 of FIG. 1A, and six electrodes 320A-320F are connected together as a “virtual ring” electrode, similar to electrode 120 of FIG. 1A. In an aspect, two individual electrodes are individually connected to a circuit to form a virtual sensor, for example electrodes 310 and 320A are respectively connected as two electrodes of a sensor. In one aspect, one or more electrodes 300 are connected together to form one or the other electrodes of a two-electrode sensor.

Any pair of electrodes, whether composed of single electrodes or a set of electrodes coupled together to form virtual electrodes, is coupled to electronics that are configured to measures an electrical property or parameter that comprises one or more of a resistance, a capacitance, an inductance, an impedance, a reluctance, or other electrical characteristic with one or more of sensors 90, 174, 290, 430, 440, or other two-electrode sensor.

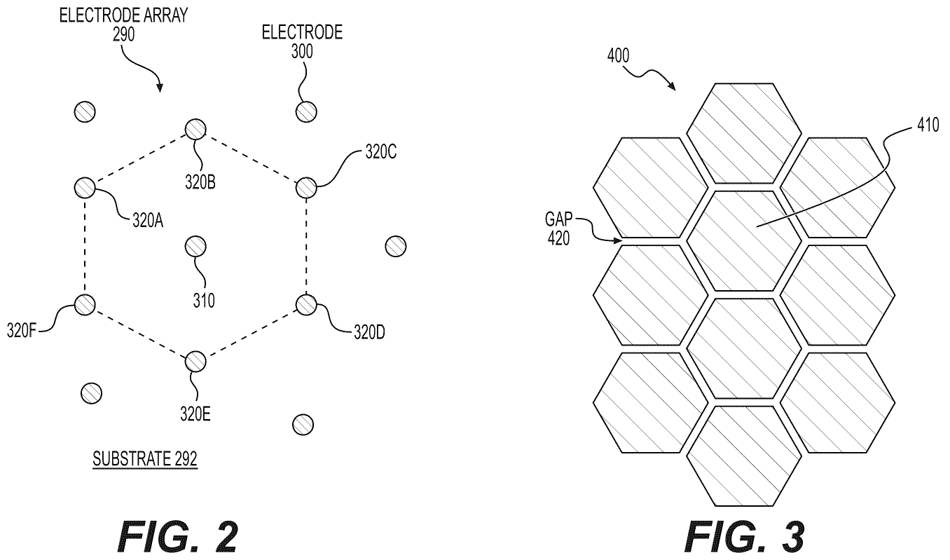

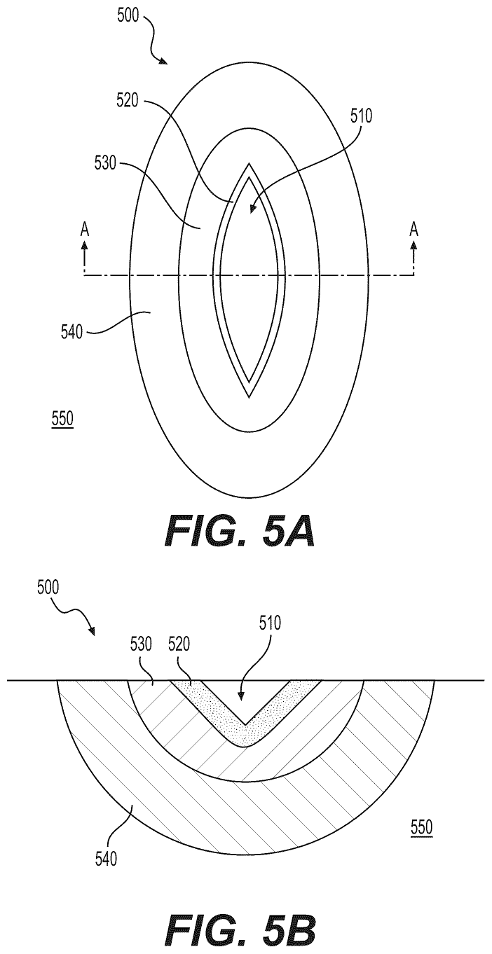

FIG. 3 depicts another exemplary array 400 of electrodes 410, according to the present disclosure. In an aspect, each of electrodes 410 is an approximate hexagon that is separated from each of the surrounding electrodes 410 by a gap 420. In one aspect, electrodes 410 are one of circles, squares, pentagons, or other regular or irregular shapes. In an aspect, gap 420 is uniform between all electrodes 410. In one aspect, gap 420 varies between various electrodes. In an aspect, electrodes 410 may be interconnected to form virtual sensors as described below with respect to FIGS. 5A and 5B.

FIG. 4A depicts an array 400 of electrodes 410 that are configured, e.g. connected to a measurement circuit, to form a sensor 430, according to the present disclosure. In an aspect, a single hexagonal electrode 410 that is labeled with a “1” forms a center electrode and a ring of electrodes 410 that are marked with a “2” are interconnected to form a ring electrode. In an aspect, electrodes 410 between the center and ring electrode are electrically “floating.” In one aspect, electrodes 410 between the center and ring electrode are grounded or connected to a floating ground. In one aspect, electrodes 410 that are outside the ring electrode are electrically “floating.” In an aspect, electrodes 410 that are outside the virtual ring electrode are grounded or connected to a floating ground.

FIG. 4B depicts an alternate aspect where an array 400 of electrodes 410 has been configured to form a virtual sensor 440, according to the present disclosure. In an aspect, multiple electrodes 410, indicated by a “1,” are interconnected to form a center electrode while a double-wide ring of electrodes, indicated by a “2,” are interconnected to form a ring electrode. In one aspect, various numbers and positions of electrodes 410 are interconnected to form virtual electrodes of a variety of sizes and shapes.

FIG. 5A depicts an example wound, in this case a 3^{rd}-degree burn 500 with an open wound 510. Response of tissue around a 3^{rd}-degree burn injury may comprise three zones. In an aspect, innermost zone 520 at the center of a wound will have necrosis with no perfusion of oxygen and irreversible damage due to the coagulation of proteins. In one aspect, second zone 530, also known as the “zone of stasis,” is a ring around a first zone 520, where there is a decrease in perfusion and a reduction in SEM. Without being limited to a particular theory, capillaries may be nonfunctional in second zone 530, leading to increased permeability of capillaries and arterioles and subsequent ischemia reperfusion injury. There may be a chance of tissue recovery in second zone 530 if cascading release of free radicals and cellular damage leading to apoptosis can be prevented. In an aspect, surrounding a second zone 530 is a zone 540 of hyperaemia where the tissue is damaged but retains good perfusion and will generally heal. Without being limited to a particular theory, the size, shape, and depth of wound 510 as well as zones 520, 530, 540 depends on the details of the event that caused the injury. In accordance with the present disclosure, evaluation of burn depth and extent is one component on which treatment decisions are based, as inaccuracies can lead to unnecessary surgeries or patients staying for extensive lengths of time.

FIG. 5B is a cross-section of a burn 500 shown in FIG. 5A, taken along line A-A in FIG. 5A. In an aspect, a first region 520 may extend below an open wound 510 as well as to the sides. In one aspect, a region 530 may extend below one or both of an open wound 510 and region 520. In an aspect, at some distance from open wound 510, there will be undamaged, or “normal,” tissue 540.

In accordance with the present disclosure, burns may be characterized as “partial thickness” or “full thickness” burns, depending upon whether damaged zones 530 and 540 extend through a skin into subcutaneous tissue. Superficial partial-thickness injuries, such as a blister of a 2nd-degree burn, are viable and will generally heal with antimicrobial dressings. Deep partial-thickness wounds are more like full-thickness burns and may require surgical excision and grafting for improved functional and cosmetic outcomes. Partial-thickness wounds are complicated to treat, as it is difficult to determine if viable structures are present and capable of healing the wound. Whatever inaccuracies associated with diagnosis may affect treatment, as it is possible that a superficial burn will receive surgery for a healing wound.

Burn wounds are challenging problems as they are dynamic and have the capacity to change and progress over time. In zone 520, heating of the tissue has caused complete necrosis of the dermis and all dermal structures along with fat necrosis. Without being limited to a particular theory, moisture content of zone 520 is lower than normal and remains low after the injury due to destruction of the local blood vessels, which prevents perfusion into the necrotic region.

Without being limited to a particular theory, in zone 530, return of blood flow after the initial thermal exposure restores perfusion and oxygenation. While not being limited to theory, the restoration of oxygenation can be important for cellular survival but also initiates a cascade of events that results in production of free radicals that lead to further tissue injury. The accumulation of burn edema can occur in a two-phase pattern. In the first phase, there is a rapid increase in interstitial fluid within the first hour post-injury and approximately 80% of total edema is present at 4 hours post-injury. The second phase is marked by a gradual increase in fluid accumulation over the next 12-24 hours. In non-burn injuries, fluid movement from the capillary to the interstitium may be generally balanced by lymphatic clearance so that excess fluid does not accumulate. However, in burn injuries, while not being limited to theory, the movement of fluid and protein into the extravascular space can occur very rapidly and edema ensues because the lymphatics are unable to keep pace with the clearance of fluid and protein. Accordingly, again without being limited to a particular theory, in an aspect, the amount of edema in zone 540 is less than in zone 530, although the amount of SEM is still increased above normal. Mapping the pattern of edema allows an assessment of which tissue is at risk.

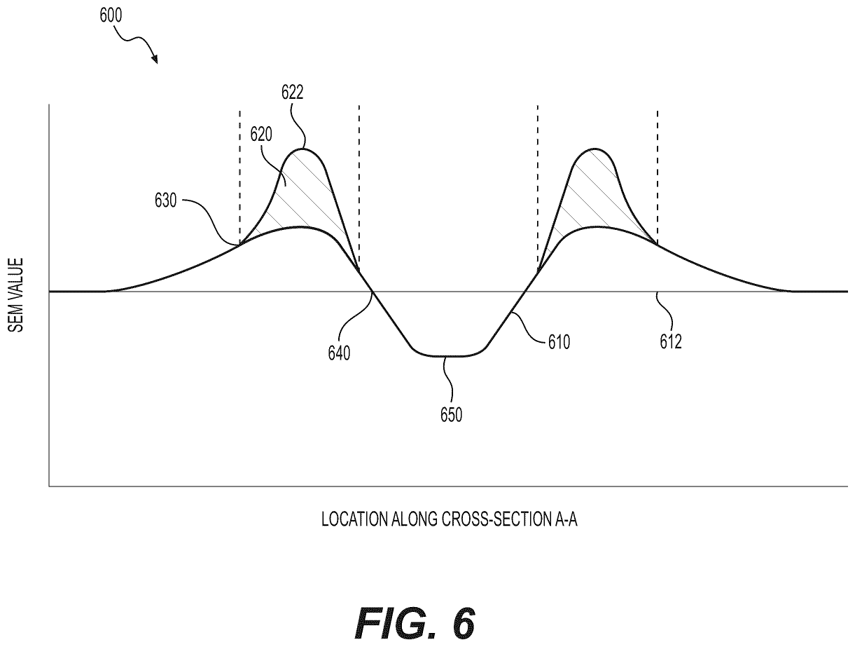

FIG. 6 depicts an example plot 600 of how SEM values may vary across burn 500, according to the present disclosure. SEM values taken along cross-section A-A have been plotted as curve 610, with the x-axis being the location along cross-section A-A and the y-axis being the SEM value. A reference line 612 indicates normal tissue SEM value, which may a standard reference value or a measurement of known undamaged tissue on the patient.

In an aspect, curve 610 generally shows a region 620 where a SEM value is greater than reference line 612. In one aspect, curve 610 in region 620 may be only slightly raised, as indicated by the bottom of the shaded region, or may be significantly increased as indicated by the top of the shaded region 620. In an aspect, a peak value 622 of region 620 is an indication of the degree or depth of the damage in zone 530.

In one aspect, point 630 on curve 610 indicates a transition from zone 530 to zone 540. In an aspect, a SEM value is higher than reference line 612 but not so elevated as to indicate a risk that a tissue will not recover. In one aspect, location of a transition from zone 530 to zone 540 may be identified on curve 610 as the x-axis position of a point 630 using a known magnitude of a SEM value. In an aspect, the magnitude of a SEM value at point 630 may be a value selected from the group consisting of a predetermined value, a predetermined increase above a reference SEM value, a percentage of a reference SEM value, a percentage of peak value 622, and other value determined from curve 610.

In an aspect, a predetermined SEM value may range from 0.1 to 8.0, such as from 0.1 to 1.0, from 1.1 to 2.0, from 2.1 to 3.0, from 3.1 to 4.0, from 4.1 to 5.0, from 5.1 to 6.0, from 6.1 to 7.0, from 7.1 to 8.0, from 0.1 to 7.5, from 0.5 to 8.0, from 1.0 to 7.0, from 1.5 to 6.5, from 2.0 to 6.0, from 3.0 to 5.5, from 3.5 to 5.0, or from 4.0 to 4.5. In an aspect, a predetermined SEM value may range from 0.1 to 4.0, such as from 0.5 to 4.0, from 0.1 to 3.5, from 1.0 to 3.5, from 1.5 to 4.0, from 1.5 to 3.5, from 2.0 to 4.0, from 2.5 to 3.5, from 2.0 to 3.0, from 2.0 to 2.5, or from 2.5 to 3.0. In one aspect, a predetermined SEM value may range from 4.1 to 8.0, such as from 4.5 to 8.0, from 4.1 to 7.5, from 5.0 to 7.5, from 5.5 to 7.0, from 5.5 to 7.5, from 6.0 to 8.0, from 6.5 to 7.5, from 6.0 to 7.0, from 6.0 to 6.5, or from 6.5 to 7.0. In one aspect, a predetermined SEM value may be about 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5. In an aspect, a predetermined SEM value can be scaled by a factor or a multiple based on the values provided herein.

In an aspect, a predetermined increase may range from 0.1 to 8.0, such as from 0.1 to 1.0, from 1.1 to 2.0, from 2.1 to 3.0, from 3.1 to 4.0, from 4.1 to 5.0, from 5.1 to 6.0, from 6.1 to 7.0, from 7.1 to 8.0, from 0.1 to 7.5, from 0.5 to 8.0, from 1.0 to 7.0, from 1.5 to 6.5, from 2.0 to 6.0, from 3.0 to 5.5, from 3.5 to 5.0, or from 4.0 to 4.5. In an aspect, a predetermined increase may range from 0.1 to 4.0, such as from 0.5 to 4.0, from 0.1 to 3.5, from 1.0 to 3.5, from 1.5 to 4.0, from 1.5 to 3.5, from 2.0 to 4.0, from 2.5 to 3.5, from 2.0 to 3.0, from 2.0 to 2.5, or from 2.5 to 3.0. In one aspect, a predetermined increase may range from 4.1 to 8.0, such as from 4.5 to 8.0, from 4.1 to 7.5, from 5.0 to 7.5, from 5.5 to 7.0, from 5.5 to 7.5, from 6.0 to 8.0, from 6.5 to 7.5, from 6.0 to 7.0, from 6.0 to 6.5, or from 6.5 to 7.0. In one aspect, a predetermined increase may be about 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5. In an aspect, a predetermined increase can be scaled by a factor or a multiple based on the values provided herein.

In one aspect, a reference SEM value is represented by a reference line 612. In an aspect, a reference SEM value may range from 0.1 to 8.0, such as from 0.1 to 1.0, from 1.1 to 2.0, from 2.1 to 3.0, from 3.1 to 4.0, from 4.1 to 5.0, from 5.1 to 6.0, from 6.1 to 7.0, from 7.1 to 8.0, from 0.1 to 7.5, from 0.5 to 8.0, from 1.0 to 7.0, from 1.5 to 6.5, from 2.0 to 6.0, from 3.0 to 5.5, from 3.5 to 5.0, or from 4.0 to 4.5. In an aspect, a reference SEM value may range from 0.1 to 4.0, such as from 0.5 to 4.0, from 0.1 to 3.5, from 1.0 to 3.5, from 1.5 to 4.0, from 1.5 to 3.5, from 2.0 to 4.0, from 2.5 to 3.5, from 2.0 to 3.0, from 2.0 to 2.5, or from 2.5 to 3.0.

In one aspect, a reference SEM value may range from 4.1 to 8.0, such as from 4.5 to 8.0, from 4.1 to 7.5, from 5.0 to 7.5, from 5.5 to 7.0, from 5.5 to 7.5, from 6.0 to 8.0, from 6.5 to 7.5, from 6.0 to 7.0, from 6.0 to 6.5, or from 6.5 to 7.0. In one aspect, a reference SEM value may be about 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5. In an aspect, a reference SEM value can be scaled by a factor or a multiple based on the values provided herein.

In an aspect, a peak value may range from 0.1 to 8.0, such as from 0.1 to 1.0, from 1.1 to 2.0, from 2.1 to 3.0, from 3.1 to 4.0, from 4.1 to 5.0, from 5.1 to 6.0, from 6.1 to 7.0, from 7.1 to 8.0, from 0.1 to 7.5, from 0.5 to 8.0, from 1.0 to 7.0, from 1.5 to 6.5, from 2.0 to 6.0, from 3.0 to 5.5, from 3.5 to 5.0, or from 4.0 to 4.5. In an aspect, a peak value may range from 0.1 to 4.0, such as from 0.5 to 4.0, from 0.1 to 3.5, from 1.0 to 3.5, from 1.5 to 4.0, from 1.5 to 3.5, from 2.0 to 4.0, from 2.5 to 3.5, from 2.0 to 3.0, from 2.0 to 2.5, or from 2.5 to 3.0. In one aspect, a peak value may range from 4.1 to 8.0, such as from 4.5 to 8.0, from 4.1 to 7.5, from 5.0 to 7.5, from 5.5 to 7.0, from 5.5 to 7.5, from 6.0 to 8.0, from 6.5 to 7.5, from 6.0 to 7.0, from 6.0 to 6.5, or from 6.5 to 7.0. In one aspect, a peak value may be about 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5. In an aspect, a peak value can be scaled by a factor or a multiple based on the values provided herein.

One or more regions may be defined on a body. In an aspect, measurements made within a region are considered comparable to each other. A region may be defined as an area on the skin of the body where measurements may be taken at any point within the area. In an aspect, a region corresponds to an anatomical region (e.g., heel, ankle, lower back). In an aspect, a region may be defined as a set of two or more specific points relative to anatomical features where measurements are taken only at the specific points. In an aspect, a region may comprise a plurality of non-contiguous areas on the body. In an aspect, the set of specific locations may include points in multiple non-contiguous areas.

In an aspect, a region is defined by surface area. In an aspect, a region may be, for example, between 5 and 200 cm², between 5 and 100 cm², between 5 and 50 cm², or between 10 and 50 cm², between 10 and 25 cm², or between 5 and 25 cm².

In an aspect, measurements may be made in a specific pattern or portion thereof. In an aspect, the pattern of readings is made in a pattern with the target area of concern in the center. In an aspect, measurements are made in one or more circular patterns of increasing or decreasing size, T-shaped patterns, a set of specific locations, or randomly across a tissue or region. In an aspect, a pattern may be located on the body by defining a first measurement location of the pattern with respect to an anatomical feature with the remaining measurement locations of the pattern defined as offsets from the first measurement position.

In an aspect, a plurality of measurements are taken across a tissue or region and the difference between the lowest measurement value and the highest measurement value of the plurality of measurements is recorded as a delta value of that plurality of measurements. In an aspect, 3 or more, 4 or more, 5 or more, 6 or more, 7 or more, 8 or more, 9 or more, or 10or more measurements are taken across a tissue or region.

In an aspect, a threshold may be established for at least one region. In an aspect, a threshold of 0.2, 0.3, 0.4, 0.5, 0.6, 0.7, 0.8, 0.9, or other value may be established for the at least one region. In an aspect, a delta value is identified as significant when the delta value of a plurality of measurements taken within a region meets or exceeds a threshold associated with that region. In an aspect, each of a plurality of regions has a different threshold. In an aspect, two or more regions may have a common threshold.

In an aspect, a threshold has both a delta value component and a chronological component, where a delta value is identified as significant when the delta value is greater than a predetermined numerical value for a predetermined portion of a time interval. In an aspect, the predetermined portion of a time interval is defined as a minimum of X days where a plurality of measurements taken that day produces a delta value greater than or equal to the predetermined numerical value within a total of Y contiguous days of measurement. In an aspect, the predetermined portion of a time interval may be defined as 1, 2, 3, 4, or 5 consecutive days on which a plurality of measurements taken that day produces a delta value that is greater than or equal to the predetermined numerical value. In an aspect, the predetermined portion of a time interval may be defined as some portion of a different specific time period (weeks, month, hours etc.).

In an aspect, a threshold has a trending aspect where changes in the delta values of consecutive pluralities of measurements are compared to each other. In an aspect, a trending threshold is defined as a predetermined change in delta value over a predetermined length of time, where a determination that the threshold has been met or exceeded is significant. In an aspect, a determination of significance will cause an alert to be issued. In an aspect, a trend line may be computed from a portion of the individual measurements of the consecutive pluralities of measurements. In an aspect, a trend line may be computed from a portion of the delta values of the consecutive pluralities of measurements.

In an aspect, the number of measurements taken within a single region may be less than the number of measurement locations defined in a pattern. In an aspect, a delta value will be calculated after a predetermined initial number of readings, which is less than the number of measurement locations defined in a pattern, have been taken in a region and after each additional reading in the same region, where additional readings are not taken once the delta value meets or exceeds the threshold associated with that region.

In an aspect, the number of measurements taken within a single region may exceed the number of measurement locations defined in a pattern. In an aspect, a delta value will be calculated after each additional reading.

In an aspect, a quality metric may be generated for each plurality of measurements. In an aspect, this quality metric is chosen to assess the repeatability of the measurements. In an aspect, this quality metric is chosen to assess the skill of the clinician that took the measurements. In an aspect, the quality metric may include one or more statistical parameters, for example an average, a mean, or a standard deviation. In an aspect, the quality metric may include one or more of a comparison of individual measurements to a predefined range. In an aspect, the quality metric may include comparison of the individual measurements to a pattern of values, for example comparison of the measurement values at predefined locations to ranges associated with each predefined location. In an aspect, the quality metric may include determination of which measurements are made over healthy tissue and one or more evaluations of consistency within this subset of “healthy” measurements, for example a range, a standard deviation, or other parameter.

In one aspect, a measurement, for example, a threshold value, is determined by SEM Scanner Model 200 (Bruin Biometrics, LLC, Los Angeles, CA). In another aspect, a measurement is determined by another SEM scanner.

In an aspect, a measurement value is based on a capacitance measurement by reference to a reference device. In an aspect, a capacitance measurement can depend on the location and other aspects of any electrode in a device. Such variations can be compared to a reference SEM device such as an SEM Scanner Model 200 (Bruin Biometrics, LLC, Los Angeles, CA). A person of ordinary skill in the art understands that the measurements set forth herein can be adjusted to accommodate a difference capacitance range by reference to a reference device.

In an aspect, a percentage in accordance with the present disclosure may range from 0-100%, such as 0-50%, 25-75%, 50-100%, 0-10%, 5-15%, 10-20%, 15-25%, 20-30%, 25-35%, 30-40%, 35%-45%, 40-50%, 0-25%, 15-35%, 25-50%, 45-55%, 50-60%, 55-65%, 60-70%, 65-75%, 40-55%, 50-75%, 70-80%, 75%-85%, 80-90%, 85-95%, 90-100%, 65-85%, or 75-100%. In one aspect, a percentage in accordance with the present disclosure may be about 0%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%.

In an aspect, point 640 on curve 610 indicates a transition from zone 530, where edema has occurred, to zone 520, where the tissue has a moisture content below normal. In one aspect, a measured SEM value that equals the normal value of reference line 612 indicates that a portion of a sensor is over tissue having a higher-than-normal moisture content while the remaining portion of the sensor is over tissue having a lower-than-normal moisture content. In an aspect, point 640 on line A-A is approximately the location of the edge of zone 520. If it is desirable to excise the necrotic tissue from a patient, marking the skin at this point provides a reference to the surgeon of the edge of necrotic tissue.

In one aspect, successive measurements of SEM values at one or more points proximate to an open wound 510, for example at 30 minute intervals for the first 4 hours, can provide information regarding the degree of damage to the tissue. In an aspect, successive measurements can be performed at approximately 5 minute intervals, 10 minute intervals, 15 minute intervals, 20 minute intervals, 25 minute intervals, 35 minute intervals, 40 minute intervals, 45 minute intervals, 50 minute intervals, 60 minute intervals, 90 minute intervals, or 120 minute intervals. In one aspect, successive measurement can be performed at time intervals for the first 1 hour, 2 hours, 3 hours, 5 hours, 6 hours, 7 hours, 8 hours, 9 hours, 10 hours, 11 hours, or 12 hours after an injury. In an aspect, the value and position of point 622 over the first 4 hours post-injury may indicate the depth of the burn and the risk of tissue depth in certain areas. Outward progression of the peak SEM values on the x-axis may indicate the severity of the reperfusion damage.

In one aspect, measurements of SEM values may be taken with a single-sensor device, such as a SEM scanner 170 of FIG. 1B, and logged, plotted, and assessed.

Other types of wounds, for example a cut, may suffer from zones of tissue death proximate to open wound 510. As the level of edema is still an indication of tissue viability, the same sensing and categorization method will provide valuable information to a clinician treating the injury. Thus, the methods and apparatus described for the example burn may also be applicable to other types of injury.

The methods and apparatus disclosed herein may also be used to track the healing process of injuries such as burns, cuts, ulcers, and other types of tissue damage. Closure of the skin over a wound is not the end of the healing process, and it may take a year after the skin closes for the sub-epidermal tissue to return to its original state. Periodic assessment of the site of the original wound will show whether the healing is continuing to progress or has halted or reversed. As an example, pressure ulcers are known to suffer a high incidence of recurrence at the same location as a first ulcer. This is thought to be a result of continued pressure at the site combined with a weakened tissue structure as a result of incomplete healing. In the absence of continued measurement of the tissue state, for example with a SEM scanner, it is likely that a caregiver would consider a closed wound as a healed wound and not continue the therapy that would prevent the recurrence. Measurements of surrounding tissue at sites away from the original wound can serve as a reference of what “normal” tissue measurements. The trend of changes, or lack thereof, of measurements at the former wound site against this reference provides a continued assurance that the tissue is moving toward a fully healed condition.

This monitoring of tissue improvement after the wound has healed is also useful to monitor the performance and efficacy of wound-healing therapies. As an example, an electro-stimulus device may be used once a wound has closed in order to accelerate the healing process of the underlying tissue. The progress of the healing is likely to be difficult if not impossible to assess manually or visually. A SEM scanning device could be used to establish one or more of a SEM measurement at the site of the closed wound, periodic measurements and trend analysis to verify the effectiveness of the healing device, and measurement of adjacent tissue as a reference of fully healed tissue. In certain embodiments, adjustments may be made to the healing device, for example a change in the frequency or voltage of an electro-stimulus device, based on the measurements or the trend of the measurements made by an SEM scanner. In certain embodiments, the use of a healing device or therapy may be halted or replaced with a different device or therapy based on the SEM measurements or trend. In certain embodiments, the wound may be judged to be “healed” based on the SEM measurement and healing therapies may be halted, modified, or replaced with preventative therapies. In certain embodiments, the difference between a current SEM reading at the site of the wound and a reference value from nearby healthy tissue is a metric of the degree of recovery of the tissue at the wound site, where a zero difference is fully healed and restored to original condition.

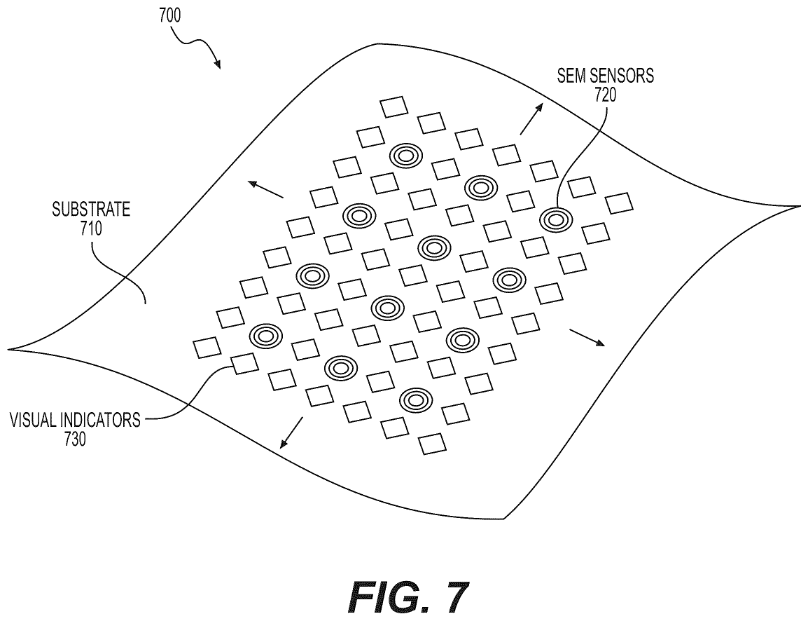

FIG. 7 depicts an aspect of a SEM sensing apparatus 700, according to the present disclosure. In one aspect, a flexible substrate 710 has a plurality of SEM sensors 720 arranged on a common surface of substrate 710. In an aspect, sensors 720 comprise toroidal sensors 90 as shown in FIG. 1A. In one aspect, sensors 720 comprise an electrode array 290 as shown in FIG. 2. In an aspect, sensors 720 comprise an electrode array 400 as shown in FIG. 3. In one aspect, sensors 720 are coupled to electronics (not shown in FIG. 7) that provide excitation and measure an SEM value of the tissue below the respective sensors 720.

In an aspect, SEM sensing apparatus 700 comprises visual indicators 730 that are arranged on a substrate 710. In one aspect, visual indicators 730 are on a first surface of a substrate 710 while sensors 720 are on a second surface of substrate 710 that is opposite the first surface. In an aspect, visual indicators 730 are disposed between at least some pairs of sensors 720. In one aspect, visual indicators 730 may be light emitting devices (LEDs). In an aspect, visual indicators 730 may emit a single color of light. In an aspect, visual indicators 730 may selectably emit one of a plurality of colors of light. In one aspect, visual indicators 730 are selectable to be on or off. In an aspect, visual indicators 730 are coupled to electronics (not shown in FIG. 7) that provide excitation and selectable control of visual indicators 730.

In an aspect, electronics of the present disclosure actuate each visual indicator 730 with a color of light selected based on the SEM values measured by sensors 90 disposed on each side of the respective visual indicator 730. This provides a color-coded map of the various zones 520, 530, and 540 for a given wound 500.

In one aspect, visual indicators 730 may be disposed on the same surface of substrate 710 as sensors 720. In an aspect, visual indicators 730 comprise marking element (not visible in FIG. 7) that can selectably mark the skin of a patient on which a SEM sensing apparatus 700 is placed. In an aspect, electronics of the present disclosure can actuate the marking element of visual indicators 720 that are disposed along one or more of the boundaries between zones of FIGS. 5A and 5B. In one aspect, electronics of the present disclosure may actuate the marking elements to mark the boundary between zone 520 and zone 530, indicating the outer edge of non-viable tissue.

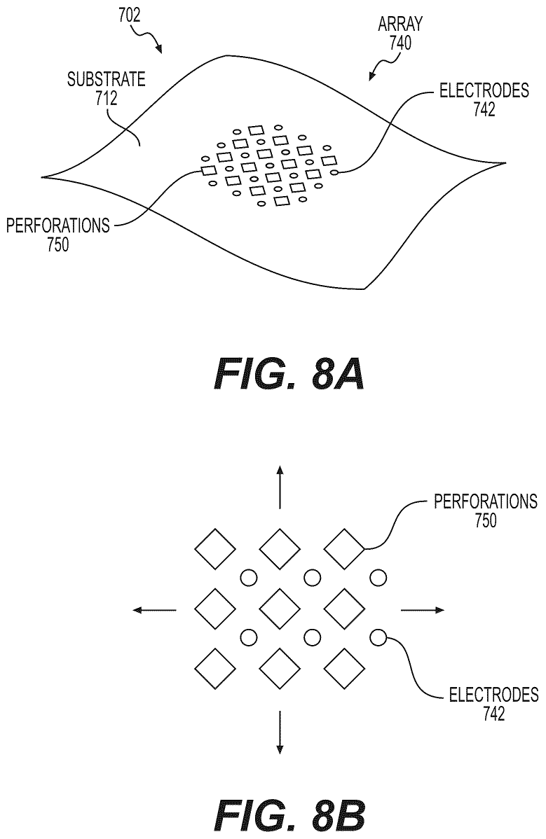

FIGS. 8A and 8B disclose an aspect of a SEM sensing assembly 702, according to the present disclosure. In one aspect, an array 740 of electrodes 742 is disposed on a substrate 712. In an aspect, electrodes 742 are similar to electrodes 300 of FIG. 2. In one aspect, electrodes 742 are similar to electrodes 410 of FIG. 3.

In an aspect, a SEM sensing apparatus 702 comprises a plurality of perforations 750. In one aspect, perforations 750 are disposed between pairs of electrodes 742, as shown in FIG. 8B. In use, SEM sensing apparatus 702 can be placed on the skin of a patient over a wound and a clinician marks the skin of the patient as guided by the SEM values measured between various pairs of electrodes 742.

In an aspect, a SEM sensing apparatus 700 may comprise both visual indicators 730 and perforations 750, allowing a clinician to mark the skin of a patient as guided by the colors of various visual indicators 730.

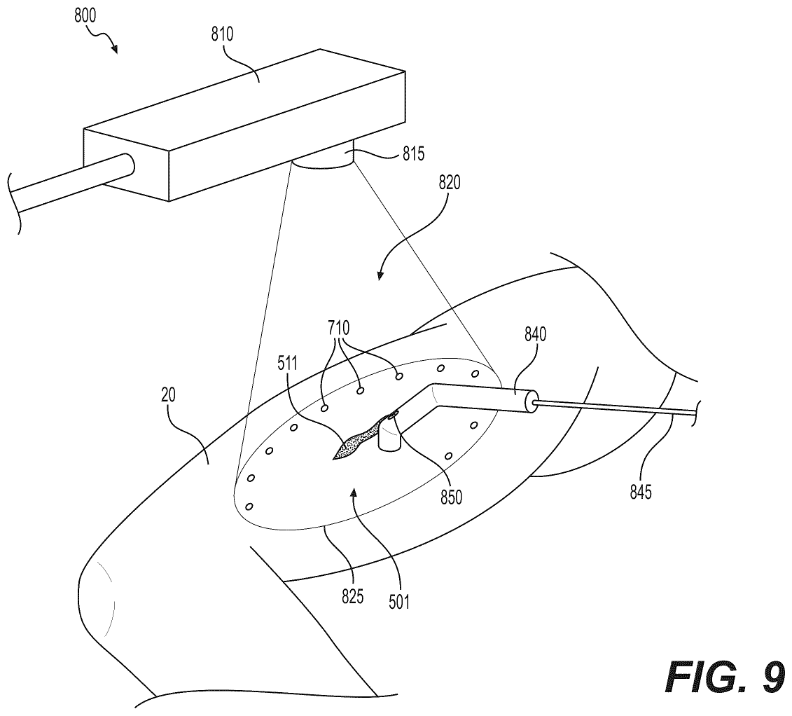

FIG. 9 discloses an aspect of an apparatus 800 for mapping areas of damage around a wound, according to the present disclosure. In one aspect, a patient's arm 20 has a burn 501 with an open wound 511. In an aspect, apparatus 800 comprises an instrument head 810 overhanging arm 20 with an optical system 815 that comprises a camera (not visible in FIG. 9) that observes area 825 on arm 20 and a projector (not visible in FIG. 9) that can project one or more images onto area 825, which encompasses wound 511 as well as tissue around wound 511. In one aspect, a SEM sensing apparatus 840 is coupled to electronics (not shown in FIG. 9) that also control optical system 815. In an aspect, SEM sensing apparatus 840 is coupled to electronics of the present disclosure through a cable 845. In an aspect, SEM sensing apparatus 840 comprises a wireless linkage in place of cable 845. In one aspect, SEM sensing apparatus 840 comprises a fiducial 850 that is visible to a camera while apparatus 800 is in use. In an aspect, SEM sensing apparatus 840 comprises a single bioimpedance sensor and, therefore, measures the ECF at a single point at a time.

In use, a user can make multiple measurements with a SEM sensing apparatus 840 in area 825. At the time of each measurement, a camera can observe and record the position of fiducial 850 in its field of view. In an aspect, reference marks (not shown in FIG. 9) may be made on arm 20 to record the position of arm 20 in the field of view and enable movement of arm 20 during an assessment. As the set of measurements increases, electronics of the present disclosure determines the location of a boundary between tissue types, for example a boundary between viable and non-viable tissue, and causes the projector to project indicating images along this boundary. In FIG. 9, these images are shown as dots 710. In an aspect, a projected image may comprise lines, areas of color, areas shading from a first color to a second color, areas shading from one intensity of a color to a different intensity of the same color, or other visual indication that provides guidance as to the condition of the tissue in area 825.

In one aspect, electronics of the present disclosure may be coupled to a printer (not shown in FIG. 9) and can cause the printer to produce a picture of arm 20 with wound 511 taken by a camera and overlaid with markings equivalent to those described as provided by a projector. In an aspect, measurements may be repeated with SEM sensing apparatus 800 and new pictures printed, thereby creating a pictorial history of the progression of damage around a wound. In one aspect, electronics of the present disclosure may be coupled to a storage device, for example a server, and configured to store information regarding an image of arm 20 and wound 511 as well as measurements and locations of the measurements made by a SEM sensing apparatus 840 at one or more times.

From the foregoing, it will be appreciated that the present invention can be embodied in various ways, which include but are not limited to the following:

Embodiment 1. An apparatus for mapping areas of damage around a wound, the apparatus comprising: a plurality of electrodes embedded on a substrate configured to be placed over an area of tissue that includes the wound, where combinations of the electrodes are capable of forming a plurality of virtual capacitive sensors and each of the virtual capacitive sensors is configured to measure a capacitance of a region of tissue proximate to the respective virtual capacitive sensor, a plurality of visual indicators embedded on the substrate, a drive circuit electronically coupled to the electrodes and visual indicators, a processor electronically coupled to the drive circuit, and a non-transitory computer-readable medium electronically coupled to the processor and comprising instructions stored thereon that, when executed on the processor, perform the steps of: receiving information regarding the measured capacitance from a subset of the plurality of virtual capacitive sensors via the drive circuit, determining a boundary between viable and non-viable tissue, and activating via the drive circuit a portion of the plurality of visual indicators to indicate the boundary.

Embodiment 2. The apparatus of embodiment 1, where the substrate comprises a plurality of perforations that allow marking of the tissue along the boundary.

Embodiment 3. The apparatus of embodiment 1, where the circuit is configured to selectively drive pairs of the electrodes and measure the capacitance between each of the pairs of electrodes.

Embodiment 4. The apparatus of embodiment 3, where each of the selectively driven pairs of electrodes form one of the plurality of virtual capacitive sensors.

Embodiment 5. The apparatus of embodiment 1, where the circuit is configured to selectively drive subsets of the plurality of electrodes to form a virtual center electrode and a virtual ring electrode and measuring the capacitance between the virtual center electrode and the virtual ring electrode.

Embodiment 6. The apparatus of embodiment 5, where each of the plurality of virtual capacitive sensors comprises a virtual center electrode and a virtual ring electrode.

Embodiment 7. The apparatus of embodiment 1, where the instructions further comprise the steps of: converting each measured capacitance to an associated sub-epidermal moisture (SEM) value that is associated with the virtual capacitive sensor used to measure the capacitance, comparing a first portion of the SEM values to a first threshold, and identifying regions of tissue corresponding to the virtual capacitive sensors that are associated with SEM values that are greater than the first threshold as viable.

Embodiment 8. The apparatus of embodiment 7, where the instructions further comprise the steps of: comparing a second portion of the SEM values to a second threshold, and identifying regions of tissue corresponding to the virtual capacitive sensors that are associated with SEM values that are less than the second threshold as non-viable.

Embodiment 9. The apparatus of embodiment 7, where each of the plurality of visual indicators independently comprises a first mode of display and a second mode of display.

Embodiment 10. The apparatus of embodiment 9, where the instructions further comprise the steps of: activating a third portion of the plurality of visual indicators in the first mode of display to indicate the regions of tissue that are viable, and activating a fourth portion of the plurality of visual indicators in the second mode of display to indicate the regions of tissue that are non-viable.

Embodiment 11. The apparatus of embodiment 9, where: the visual indicators are light-emitting devices (LEDs), the first mode of display comprises emitting light having a first characteristic, and the second mode of display comprises emitting light having a second characteristic.

Embodiment 12. The apparatus of embodiment 11, where: the first characteristic comprises a first spectral content, and the second characteristic comprises a second spectral content that is different from the first spectral content.

Embodiment 13. An apparatus for determining a depth of a burn wound, the apparatus comprising: a pair of electrodes capable of forming a capacitive sensor that is configured to measure a capacitance of a region of tissue proximate to the pair of electrodes, a drive circuit electronically coupled to the capacitive sensor, a processor electronically coupled to the drive circuit, and a non-transitory computer-readable medium electronically coupled to the processor and comprising instructions stored thereon that, when executed on the processor, perform the steps of: receiving information regarding the measured capacitance from the capacitive sensor via the drive circuit, comparing the information to a data array comprising pairs of capacitances and depths of burns, and determining the depth of the burn wound associated with the measured capacitance.

Embodiment 14. The apparatus of embodiment 13, where: the step of receiving information regarding the measured capacitance comprises: receiving a first capacitance measured at a first location of known unaffected tissue, receiving a second capacitance measured at a second location within the burn wound, and determining a capacitance difference between the first and second capacitances; the data array comprises pairs of capacitance differences and depths of burns; the step of comparing the information to the data array comprises comparing the capacitive difference to the data array; and the step of determining the depth of the burn wound comprises identifying the depth of the burn wound associated with the capacitive difference.

Embodiment 15. The apparatus of embodiment 13, where: the instructions further comprise the step of converting each measured capacitance to an associated sub-epidermal moisture (SEM) value, the data array comprises pairs of SEM values and depths of burns, the step of comparing the information to the data array comprises comparing the SEM value to the data array, and the step of determining the depth of the burn wound comprises identifying the depth of the burn wound associated with the SEM value.

Embodiment 16. An apparatus for mapping areas of damage around a wound, the apparatus comprising: a plurality of electrodes embedded on a substrate configured to be placed over a portion of an area of tissue that includes the wound, where pairs of the electrodes are capable of forming a capacitive sensor that is configured to measure a capacitance of a region of tissue proximate to the capacitive sensor, a projector capable of projecting a visual indicator onto the area of tissue that includes the wound, a drive circuit electronically coupled to the plurality of electrodes and the projector, a processor electronically coupled to the drive circuit, and a non-transitory computer-readable medium electronically coupled to the processor and comprising instructions stored thereon that, when executed on the processor, perform the steps of: receiving information regarding the measured capacitance from one or more of the formed capacitive sensors, determining a first boundary between a first type of tissue and a second type of tissue, and causing the projector to project the visual indicator to indicate the boundary.

Embodiment 17. The apparatus of embodiment 16, where the first type of tissue is a viable tissue, and the second type of tissue is a non-viable tissue.

Embodiment 18. The apparatus of embodiment 17, where the first boundary is identified by: converting each measured capacitance to an associated sub-epidermal moisture (SEM) value that is associated with the capacitive sensor used to measure the capacitance, identifying regions of tissue corresponding to the capacitive sensors that are associated with SEM values that are greater than a threshold as viable, identifying regions of tissue corresponding to the capacitive sensors that are associated with SEM values that are lesser than the threshold as non-viable; and marking a first boundary between the viable and non-viable regions.

Embodiment 19. The apparatus of embodiment 16, where the instructions further comprise the step of determining a second boundary between the second type of tissue and a third type of tissue.

Embodiment 20. The apparatus of embodiment 19, where the first type of tissue is a necrotic tissue, where the second type of tissue is a tissue in a zone of stasis, and where the third type of tissue is a tissue in a zone of hyperaemia.

Embodiment 21. The apparatus of embodiment 20, where the first and second boundaries are identified by: converting each measured capacitance to an associated sub-epidermal moisture (SEM) value that is associated with the capacitive sensor used to measure the capacitance, identifying regions of tissue corresponding to the capacitive sensors that are associated with SEM values that are lesser than a first threshold as a necrotic tissue, marking the first boundary on an outer edge of the necrotic tissue regions, identifying regions of tissue in a zone of stasis comprising tissue immediately surrounding the regions of necrotic tissue and corresponding to the capacitive sensors that are associated with SEM values that are greater than the first threshold up to and including locations associated with a peak SEM value, and tissue immediately surrounding the locations associated with the peak SEM value and corresponding to the capacitive sensors that are associated with SEM values that are greater than a second threshold, marking the second boundary on an outer edge of the zone of stasis; and identifying regions of tissue in a zone of hyperaemia comprising tissue immediately surrounding the zone of stasis and corresponding to the capacitive sensors that are associated with SEM values that are lesser than the second threshold but greater than the first threshold.

Embodiment 22. A method for mapping areas of damage around a wound, the method comprising: obtaining capacitance measurements over an area of a tissue including the wound using a plurality of electrodes; converting each measured capacitance to an associated sub-epidermal moisture (SEM) value; and marking a first boundary encompassing regions of tissue associated with SEM values that are lesser than a first threshold.

Embodiment 23. The method of embodiment 22, further comprising: marking a second boundary surrounding the first boundary and encompassing regions of tissues associated with SEM values that are greater than the first threshold up to and including locations associated with a peak SEM value, and tissue immediately surrounding the locations associated with the peak SEM value and are associated with SEM values that are greater than a second threshold.

While the invention has been described with reference to particular aspects, it will be understood by those skilled in the art that various changes may be made and equivalents may be substituted for elements thereof without departing from the scope of the invention. In addition, many modifications may be made to a particular situation or material to the teachings of the invention without departing from the scope of the invention. Therefore, it is intended that the invention not be limited to the particular aspects disclosed but that the invention will include all aspects falling within the scope and spirit of the appended claims.

1. An apparatus for mapping areas of damage around a wound, said apparatus comprising:
a plurality of electrodes embedded on a substrate configured to be placed over an area of tissue that includes the wound, wherein combinations of said electrodes are capable of forming a plurality of virtual capacitive sensors and each of said virtual capacitive sensors is configured to measure a capacitance of a region of tissue proximate to said respective virtual capacitive sensor,
a plurality of visual indicators embedded on said substrate,
a drive circuit electronically coupled to said electrodes and visual indicators,
a processor electronically coupled to said drive circuit, and
a non-transitory computer-readable medium electronically coupled to said processor and comprising instructions stored thereon that, when executed on said processor, perform the steps of:
receiving information regarding said measured capacitance from a subset of said plurality of virtual capacitive sensors via said drive circuit,
determining a boundary between viable and non-viable tissue, and
activating via said drive circuit a portion of said plurality of visual indicators to indicate said boundary.

2. The apparatus of claim 1, wherein said substrate comprises a plurality of perforations that allow marking of said tissue along said boundary.

3. The apparatus of claim 1, wherein said drive circuit is configured to selectively drive pairs of said electrodes and measure said capacitance between each of said pairs of electrodes.

4. The apparatus of claim 3, wherein each of said selectively driven pairs of electrodes form one of said plurality of virtual capacitive sensors.

5. The apparatus of claim 1, wherein said circuit is configured to selectively drive subsets of said plurality of electrodes to form a virtual center electrode and a virtual ring electrode and measuring said capacitance between said virtual center electrode and said virtual ring electrode.

6. The apparatus of claim 5, wherein each of said plurality of virtual capacitive sensors comprises a virtual center electrode and a virtual ring electrode.

7. The apparatus of claim 1, wherein said instructions further comprise the steps of:
converting each measured capacitance to an associated sub-epidermal moisture (SEM) value that is associated with said virtual capacitive sensor used to measure said capacitance,
comparing a first portion of said SEM values to a first threshold, and
identifying regions of tissue corresponding to said virtual capacitive sensors that are associated with SEM values that are greater than said first threshold as viable.

8. The apparatus of claim 7, wherein said instructions further comprise the steps of:
comparing a second portion of said SEM values to a second threshold, and
identifying regions of tissue corresponding to said virtual capacitive sensors that are associated with SEM values that are less than said second threshold as non-viable.

9. The apparatus of claim 7, wherein each of said plurality of visual indicators independently comprises a first mode of display and a second mode of display.

10. The apparatus of claim 9, wherein said instructions further comprise the steps of:
activating a third portion of said plurality of visual indicators in said first mode of display to indicate the regions of tissue that are viable, and
activating a fourth portion of said plurality of visual indicators in said second mode of display to indicate the regions of tissue that are non-viable.

11. The apparatus of claim 9, wherein:
said visual indicators are light-emitting devices (LEDs),
said first mode of display comprises emitting light having a first characteristic, and said second mode of display comprises emitting light having a second characteristic.

12. The apparatus of claim 11, wherein:
said first characteristic comprises a first spectral content, and
said second characteristic comprises a second spectral content that is different from said first spectral content.

13. An apparatus for determining a depth of a burn wound, said apparatus comprising:
a pair of electrodes capable of forming a capacitive sensor that is configured to measure a capacitance of a region of tissue proximate to said pair of electrodes,
a drive circuit electronically coupled to said capacitive sensor,
a processor electronically coupled to said drive circuit, and
a non-transitory computer-readable medium electronically coupled to said processor and comprising instructions stored thereon that, when executed on said processor, perform the steps of:
receiving information regarding said measured capacitance from said capacitive sensor via said drive circuit,
comparing said information to a data array comprising pairs of capacitances and depths of burns, and
determining the depth of the burn wound associated with said measured capacitance.

14. The apparatus of claim 13, wherein:
said step of receiving information regarding said measured capacitance comprises:
receiving a first capacitance measured at a first location of known unaffected tissue,
receiving a second capacitance measured at a second location within said burn wound, and
determining a capacitance difference between said first and second capacitances;
said data array comprises pairs of capacitance differences and depths of burns;
said step of comparing said information to said data array comprises comparing said capacitive difference to said data array; and
said step of determining the depth of said burn wound comprises identifying the depth of said burn wound associated with said capacitive difference.

15. The apparatus of claim 13, wherein:
said instructions further comprise the step of converting each measured capacitance to an associated sub-epidermal moisture (SEM) value,
said data array comprises pairs of SEM values and depths of burns,
said step of comparing said information to said data array comprises comparing said SEM value to said data array, and
said step of determining the depth of said burn wound comprises identifying the depth of said burn wound associated with said SEM value.

16. An apparatus for mapping areas of damage around a wound, said apparatus comprising:
a plurality of electrodes embedded on a substrate configured to be placed over a portion of an area of tissue that includes said wound, wherein pairs of said electrodes are capable of forming a capacitive sensor that is configured to measure a capacitance of a region of tissue proximate to said capacitive sensor,
a projector capable of projecting a visual indicator onto said area of tissue that includes said wound,
a drive circuit electronically coupled to said plurality of electrodes and said projector,
a processor electronically coupled to said drive circuit, and
a non-transitory computer-readable medium electronically coupled to said processor and comprising instructions stored thereon that, when executed on said processor, perform the steps of:
receiving information regarding said measured capacitance from one or more of said formed capacitive sensors,
determining a first boundary between a first type of tissue and a second type of tissue, and causing said projector to project said visual indicator to indicate said boundary.

17. The apparatus of claim 16, wherein said first type of tissue is a viable tissue, and said second type of tissue is a non-viable tissue.

18. The apparatus of claim 17, wherein said first boundary is identified by:
converting each measured capacitance to an associated sub-epidermal moisture (SEM) value that is associated with said capacitive sensor used to measure said capacitance,
identifying regions of tissue corresponding to said capacitive sensors that are associated with SEM values that are greater than a threshold as viable,
identifying regions of tissue corresponding to said capacitive sensors that are associated with SEM values that are lesser than said threshold as non-viable; and
marking a first boundary between the viable and non-viable regions.

19. The apparatus of claim 16, wherein said instructions further comprise the step of determining a second boundary between said second type of tissue and a third type of tissue.

20. The apparatus of claim 19, wherein said first type of tissue is a necrotic tissue, wherein said second type of tissue is a tissue in a zone of stasis, and wherein said third type of tissue is a tissue in a zone of hyperaemia.

21-23. (canceled)
